(19) United States
(12) Patent Application Publication
(10) Pub. No.: US 2020/0268319 A1
(43) Pub. Date: Aug. 27, 2020

(54) JOINT FUNCTION MONITORING SYSTEM

(71) Applicant: Conzian Ltd., Taipei (TW)

(72) Inventors: YAN-FU LIU, Taipei (TW); PO-JUI HUANG, Taipei (TW); CHUNG-HSIEN WU, Taipei (TW); JYUN-AN YAO, Taipei (TW)

(21) Appl. No.: 16/286,697

(22) Filed: Feb. 27, 2019

(51) Int. Cl. A61B 5/00; A61B 5/11; A61F 5/01

(52) U.S. Cl. CPC A61B 5/6812; A61B 5/1121; A61B 5/4571; A61B 5/4576; A61B 5/458; A61B 2505/09; A61B 5/459; A61B 5/4595; A61F 5/0123; A61F 5/0127; A61F 5/013; A61B 5/4585

(57) ABSTRACT

The present disclosure provides a joint function monitoring device. The joint function monitoring device comprises a bottom base capable of being removably attached to a hinge of an assistive device, a shaft comprising a central pan coupled to the bottom base, and a distal part, a first sensor coupled to the central part of the shaft for monitoring a rotation angle of the shaft, and a belt module coupled to the distal pan of the shaft and capable of being removably attached to a support assembly of the assistive device. A length of the belt module is adjustable.

### FIELD OF THE INVENTION

The present disclosure relates to a joint function monitoring system. Specifically, the present disclosure relates to a joint function monitoring device for the joint function monitoring system.

### BACKGROUND OF THE INVENTION

Rehabilitation are therapeutic approaches for restoring functions of one or more parts of human body. The rehabilitation of joint aims to restore the function of joints after injury, impairment, invasive treatment, and surgery. These conditions may include: neuromuscular damage, musculoskeletal damage, degenerative disease, joint replacement procedures, repairing bone fracture of the arm or the leg, or arthroscopic surgeries for the knee or the hip.

Joint orthoses are commonly used in joint rehabilitation for assisting the movement and providing protection for the joint. The joint orthoses can be a joint brace or a joint splint. Knee braces are one of the many commercially available joint orthoses for the rehabilitation of the knee joint. The EPO™ Post-Op Knee Brace manufactured by Breg, Inc. is a post-operative knee brace with adjustable tabs to fit different leg sizes. The EPO™ Post-Op Knee Brace includes a hinge with extension and flexion locks to limit the movement of the knee, and at least two support assemblies extending from the hinge to be attached to the patients' leg. During the rehabilitation process wherein the patient is wearing the knee brace, the rehabilitation specialist and the physician may use the extension and flexion locks.

After the knee braces are worn by the patient, it would require the rehabilitation specialist and the physician to examine movements of the knee, in order to evaluate the rehabilitation. However, knee movement records from the patient's notes, retrospective interviews, and appointments may be incorrect. Therefore, a real-time monitoring and analysis of knee movements are required to provide correct records of the knee movement.

To retrieve knee movement information, some knee braces are combined with sensors. A knee brace combined with electrical stimulation module, sensors, and feedback mechanism is proposed in Mavroidis, C. et al (Smart portable rehabilitation devices. Journal of NeuroEngineering and Rehabilitation (2005) 2(1), 18). The electrical stimulation information is transmitted to a control box to monitor the muscle activity of the knee, and to provide additional electrical stimulation when possible.

X4™ Smart Brace is a supportive knee brace that monitors the movements of the knee in real-time. X4™ Smart Brace is equipped with a sensor for monitoring flexion and extension of the knee, and a transmission module to transmit a series of flexion or extension information to an external device, and the external device can be a smartphone or a computer. The external device then stores the information for further evaluation.

The solution proposed by Mavroidis, C. et al and X4™ Smart Brace incorporate the sensor in the knee brace. However, for a plus-size patient or a pediatric patient, if the sensor-incorporated knee brace does not fit, then this sensor-integrated knee brace would not be applicable for these patients. Also, if a patient wish to switch to the sensor-integrated knee brace from a knee brace without sensors, the patient may need to purchase a brand-new sensor-integrated knee brace and discard the previous knee brace. These are factors that may increase the cost of the joint rehabilitation for the patient.

### BRIEF SUMMARY OF THE INVENTION

It is an object of the present disclosure to provide a joint function monitoring device that can be removably attached to a joint orthosis. The modular design of the joint function monitoring device provides greater flexibility for the patient and can be installed onto joint orthoses of various shapes and sizes.

It is also an objective of the present disclosure to provide a joint function monitoring device that measures and analyzes joint movements in real-time.

It is also an object of the present disclosure to provide a joint function monitoring system comprising an assistive device for supporting a joint and a joint function monitoring device for sensing movements of the joint.

An embodiment of the present disclosure provides a joint function monitoring device. The joint function monitoring device comprises a bottom base capable of being removably attached to a hinge of an assistive device, a shaft comprising a central part coupled to the bottom base, and a distal pan, a first sensor coupled to the central part of the shaft for monitoring a rotation angle of the shaft, and a belt module coupled to the distal part of the shaft and capable of being removably attached to a support assembly of the assistive device. Wherein a length of the belt module is adjustable.

In a preferred embodiment, the belt module comprises one or more links and a link base on a distal end of the belt module, the link base is coupled to at least one of the links and the support assembly of the assistive device, and at least one of the links is removably coupled to the distal part of the shaft.

In a preferred embodiment, the amount of the links is in a range of one to six.

In a preferred embodiment, the joint function monitoring device further comprises a housing coupled to the bottom base, and a control module accommodated by the housing.

In a preferred embodiment, the control module further comprises a controller communicatively coupled to the first sensor, and a second sensor communicatively coupled to the controller.

In a preferred embodiment, the joint function monitoring device further comprises a locator, when the bottom base is attached to the hinge of the assistive device, the locator is beneath the housing and aligned with the hinge.

In a preferred embodiment, the shaft further comprises an axis on the central part, and a center of the rotation of the shaft is the axis, and the rotation angle is about 0°-360°.

In a preferred embodiment, the axis is coupled to the first sensor.

Another embodiment of the present disclosure provides a joint function monitoring system. The joint function monitoring system comprises an assistive device and a joint function monitoring device. The assistive device comprises a hinge capable of being aligned with a joint of a mammalian body, and a support assembly capable of being attached to a part of the mammalian body adjacent to the joint. The joint function monitoring device comprises a bottom base removably attached to the hinge of the assistive device, a shaft comprising a central part coupled to the bottom base and a distal part, a first sensor coupled to the central part of the shaft for monitoring a rotation angle of the shaft, and a belt module coupled to the distal part of the shaft and capable of being removably attached to the support assembly of the assistive device. Wherein a length of the belt module is adjustable.

In a preferred embodiment, the joint is an ankle joint, a knee joint, a hip joint, a shoulder joint, an elbow joint, or a wrist joint.

In a preferred embodiment, the assistive device is an ankle orthosis when the joint is the ankle joint, a knee orthosis when the joint is the knee joint, a hip orthosis when the joint is the hip joint, a shoulder orthosis when the joint is the shoulder joint, or a wrist orthosis when the joint is the wrist joint.

In a preferred embodiment, the pan of the mammalian body adjacent to the joint can be a foot, a leg, a hip, a shoulder, an arm, or a hand.

### BRIEF DESCRIPTION OF THE DRAWINGS

The accompanying drawings illustrate one or more embodiments of the present disclosure and, together with the written description, explain the principles of the present disclosure. Wherever possible, the same reference numbers are used throughout the drawings referring to the same or like elements of an embodiment.

FIG. 1 is a perspective view of a first joint function monitoring system, in accordance with an embodiment of the present disclosure.

FIG. 2 is a perspective view of an assistive device, in accordance with an embodiment of the present disclosure.

FIG. 3 is a perspective view of a joint function monitoring device, in accordance with an embodiment of the present disclosure.

FIG. 4 is an exploded view of the joint function monitoring device, in accordance with an embodiment of the present disclosure.

FIG. 5 is an exploded view of some of the elements of the joint function monitoring device, in accordance with an embodiment of the present disclosure.

FIG. 6 is another exploded view of some of the elements of the joint function monitoring device, in accordance with an embodiment of the present disclosure.

FIG. 7 is a side view of some of the elements of the joint function monitoring device, in accordance with an embodiment of the present disclosure.

FIGS. 8A-8D are illustrations of a first part of an installation process for the joint function monitoring device to be attach d onto the assistive device, in accordance with an embodiment of the present disclosure.

FIGS. 9A-9D are illustrations of a second part of the installation process for the joint function monitoring device to be attached onto the assistive device, in accordance with an embodiment of the present disclosure.

FIGS. 10A and 10B are illustrations of an extension and a flexion of the first joint function monitoring system, in accordance with an embodiment of the present disclosure.

FIG. 11 is a perspective view of a second joint function monitoring system, in accordance with an embodiment of the present disclosure.

FIG. 12 is a perspective view of another assistive device, in accordance with embodiment of the present disclosure.

FIG. 13 is a perspective vies of a third joint function monitoring system, in accordance with an embodiment of the present disclosure.

FIG. 14 is a perspective view of a fourth joint function monitoring system worn by a subject, in accordance with an embodiment of the present disclosure.

FIG. 15 is a perspective view of a fifth joint function monitoring system worn by the subject, in accordance with an embodiment of the present disclosure.

In accordance with common practice, the various described features are not drawn to scale and are drawn to emphasize features relevant to the present disclosure. Like reference characters denote like elements throughout the figures and text.

### DETAILED DESCRIPTION OF THE PREEFERRED EMBODIMENTS

The present disclosure will now be described more fully hereinafter with reference to the accompanying drawings illustrating various exemplary embodiments of the invention. The present disclosure may, however, be embodied in many different forms and should not be construed as limited to the embodiments set forth herein. Rather, these embodiments provided so that this disclosure will be thorough and complete and will fully the scope of the disclosure to those skilled in the art. Like reference numerals refer to like elements throughout.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

The term “subject” refer to an individual mammalian wearing one part or all parts of the devices provided by the present disclosure. The terms “physician, ” “rehabilitation specialist,” and “user” are interchangeable between each other, and refer to one or more individuals assisting the subject to wear one part or all parts of the devices provided by die present disclosure. The user, physician, or rehabilitation specialist may evaluate physical rehabilitation outcome of the subject by using the devices provided by the present disclosure. The “subject” and the “user” can be different individuals or the same individual.

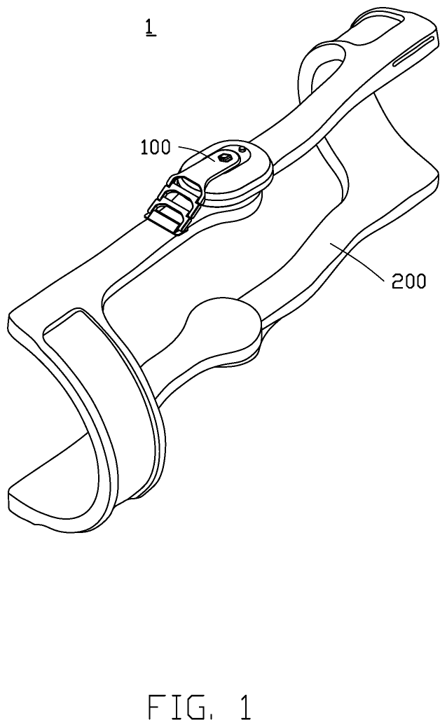

Referring to FIG. 1, a first joint function monitoring system is provided in accordance with an embodiment of the present disclosure. The first joint function monitoring system 1 comprises an assistive device 200 and a joint function monitoring device 100 attached on the assistive device 200. The assistive device 200 can be worn by a mammalian subject, and corresponds to shapes of a joint and body parts adjacent to the joint of the mammalian subject. The joint can be an ankle joint, a knee joint, a hip joint, a shoulder joint, an elbow joint, or a wrist joint. The body parts adjacent to the joint can be a foot, a leg, a hip. a shoulder, an arm, or a hand. The assistive device 200 is able to assist movements of the joint and the adjacent body parts, such as extension, flexion, or rotation. The assistive device 200 can be an ankle orthosis when the joint is the ankle joint, a knee orthosis when the joint is the knee joint, a hop orthosis when the joint is the hip joint, a shoulder orthosis when the joint is the shoulder joint, or a wrist orthosis when the joint is the wrist joint. The joint orthosis can be a joint brace or a joint sprint. Preferably, the assistive device 200 is a knee brace. The joint function monitoring device 100 can be manually removed or attached onto the assistive device 200 by the mammalian subject, therefore it is removably attached on the assistive device 200.

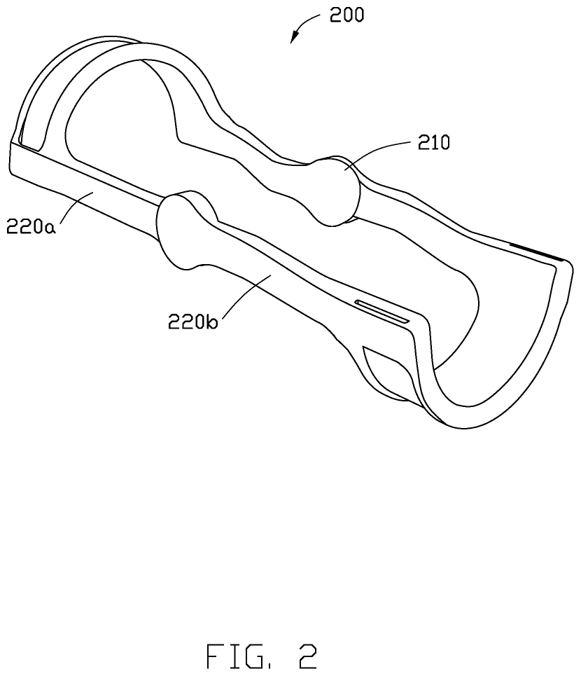

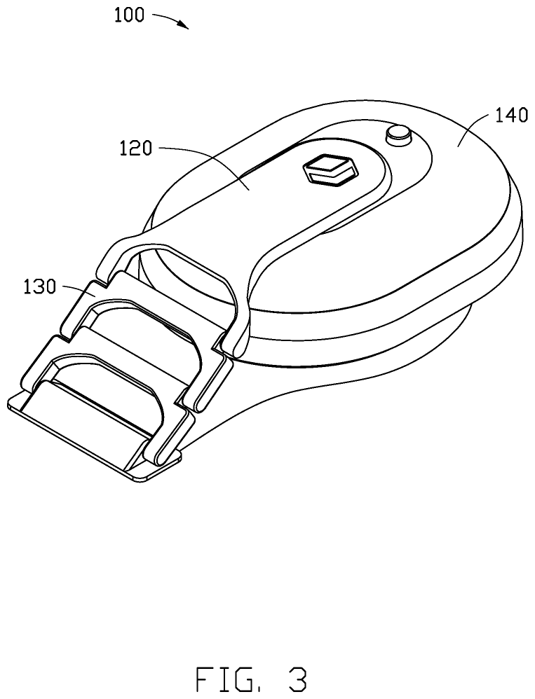

Referring to FIG. 2, the assistive device 200 is provided in accordance with an embodiment of the present disclosure. The assistive device 200 comprises a hinge 210 and 2 support assemblies 220a and 220b coupled to an upper part and a lower part of the hinge 210. The support assemblies 220a and 220b are fixation elements of the assistive device 200, and may comprise bars or bands. The bars or bands of the support assemblies 220a and 220b may have an elongated shape to conform with the shape of the body parts adjacent to the joint, The support assemblies 220a and 220b may be attached to the body part adjacent to the joint by Velcro®, buttons, or straps, therefore the assistive device 200 can be firmly worn by the mammalian subject. The support assemblies 220a and 220b can be made of soft, semi-rigid, or rigid materials that is durable but also able to sustain persistent contact with the subject's skin. The hinge 210 provides extension or flexion movements within the assistive device 200, and may comprise gears or hydraulic devices for the extension or flexion movements. When the assistive device 200 is worn by the mammalian subject, the hinge 210 is aligned with the joint. Thus, when the mammalian subject bend the joint, the assistive device 200 is bended, and the support assemblies 220a and 220b will not form a straight line as illustrated in FIG. 2. The assistive device 200 can be composed of plastic material such as polyoxymethylene (POM), metal materials such as steel or aluminum alloy, or a combination of plastic and metal materials. The assistive device 200 should be sturdy enough to withstand the movement of the joint without strain or failure.

Referring to FIG, 3, the joint function monitoring device 100 is provided in accordance with an embodiment of the present disclosure. The joint function monitoring device 100 comprises a housing 140, a shaft 120 attached on the housing 140, a first sensor (not shown) coupled to the shaft 120, and a belt module 130 also coupled to the shaft 120. The housing 140 accommodates several internal components of the joint function monitoring device 100 and provides protection to the internal components. The shaft 120 is rotatable relative to the housing 140. The belt module 130 can be coupled to the support assembly 220a or 220b of the assistive device 200.

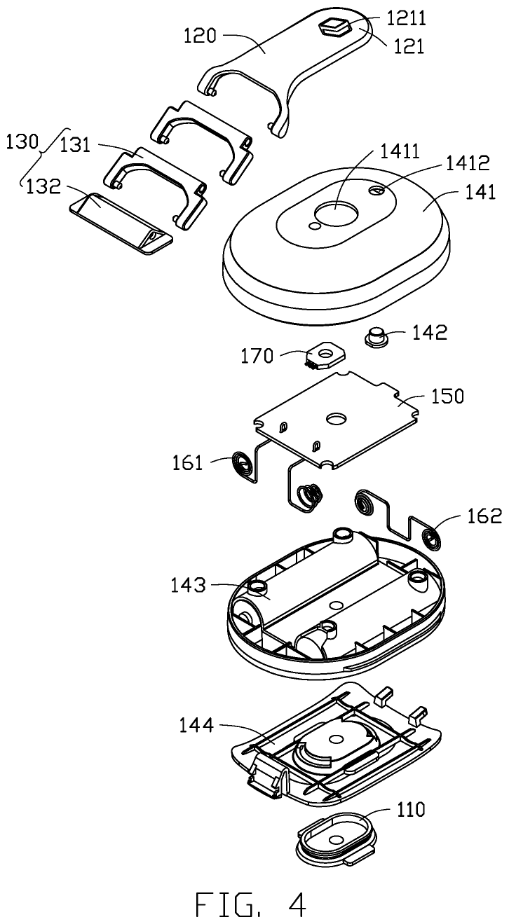

Referring to FIG. 4, an exploded view of the joint function monitoring device 100 is provided in accordance with an embodiment of the present disclosure. The belt module 130 comprises a link base 132 and a link 131 coupled to the link base 132. The link base 132 is installed onto the support assembly 220a or 220b. Specifically, the link base 132 is removably attache d to the support assembly 220a or 220b. The belt module 130 may comprise more than one link 131, depending on the shape or size of the assistive device 200. The housing 140 comprises an upper housing 141 having an opening 1411 and a hole 1412 and covering an upper side and a part of a lateral side of the joint unction monitoring device 100, a button 142 for a user to interact with the internal components accommodated inside the housing 140 and fits into the hole 1412 of the upper housing 141, a middle housing 143 providing spaces to accommodate batteries and covering another part of the lateral side and a part of a bottom side of the joint function monitoring device 100, and a bottom 144 supporting the batteries and covering most of the bottom side of the joint function monitoring device 100 and contacting the assistive device 200 when the joint function monitoring device 100 is attached to the assistive device 200. The upper housing 141 generally has an egg-shape, an elliptical shape, or a compound shape of a rectangle and two semi-circle, and is curved on an edge. To conform with the shape of the upper housing 141, the middle housing 143 may also have an egg-shape, an elliptical shape, or the compound shape of a rectangle and two semi-circle.

The shaft 120 comprises a central pan 121 for coupling with the upper housing 141, Specifically, the central part 121 of the shaft 120 comprises a coupling structure 1211 that is complementary to the opening, 1411 of the upper housing 141. The central part 121 further comprises an axis (not shown) extending downwardly from the coupling structure 1211 and goes through the opening 1411 of the upper housing 141. The first sensor 170 is coupled to the axis of the central part 121 and housed by the upper housing 141.

The bottom 144 can be removed by the user to install or remove the batteries. A battery circuit 161 has electrical connection with the batteries and is coupled to a control module 150. Another battery circuit 162 also has electrical connection with the batteries and is coupled to the middle housing 143. A bottom base 110 is capable of being removably attached to the hinge 210 of the assistive device 200.

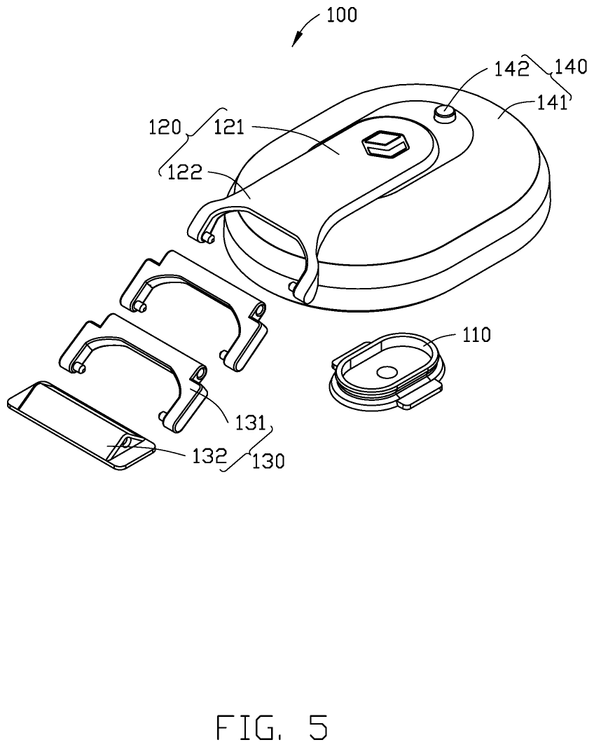

Referring to FIG. 5, an exploded view of the joint function monitoring device 100 with some of the elements is provided in accordance with an embodiment of the present disclosure. When the user is assembling the joint function monitoring device 100, the upper housing 141, the button 142, the control module 150 (not shown), the battery circuit 161 and 162, the middle housing 143 (not shown), the bottom 144 (not shown), the first sensor 170 (not shown), and shaft 120 are combined into a modular piece; wherein the bottom base 110, the link 131, and the link base 132 are usually separated from the modular piece. The button 142 is protruding from the hole 1412 of the upper housing 141 and the user or the subject may press the button 142 to interact with the internal components, preferably to interact with the control module 150. The shaft 120 comprises the central part 121 coupled to the opening 1411 of the upper housing 141 and a distal part 122 extending from the central part 121.

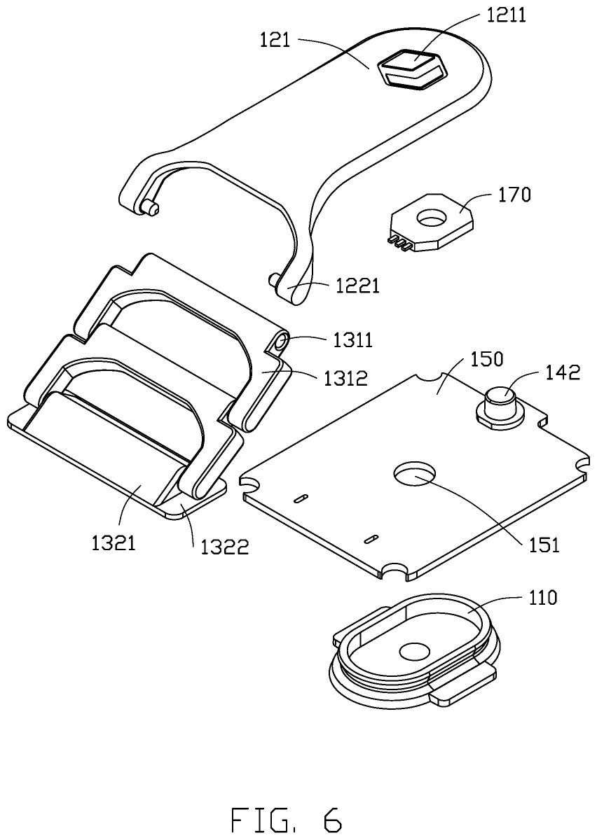

Referring to FIG. 6, another exploded view of the joint function monitoring device 100 with some of the elements is provided in accordance with an embodiment of the present disclosure. In FIG. 6, the upper housing 141, the middle housing 143, and the bottom 144 are omitted to show details of other elements. The coupling, structure 1211 is complementary to an opening 151 at a center of the control module 150. The distal part 122 comprises a fork end 1221 that is neighboring the edge of the upper housing 141 and is structurally complementary to a plurality of receiving portions 1311 of the link 131. Similarly, the link 131 also comprises a fork end 1312 that is structurally complementary to a receiving portion 1321 of the link, base 132, The link base 132 further comprises an attachment portion 1322 for being removably attached to the support assembly 220a or 220b of the assistive device 200.

The fork end 1312 of the link 131 may have two protrusions and the receiving portion 1321 of the link base 132 may have two recesses to accommodate the protrusions of the fork end 1312, as illustrated in FIG. 6. Conversely, the fork end 1312 of the link 131 may have two recesses and the receiving portions 1321 of the link base 132 may have two protrusions to insert into the recesses of the fork end 1312.

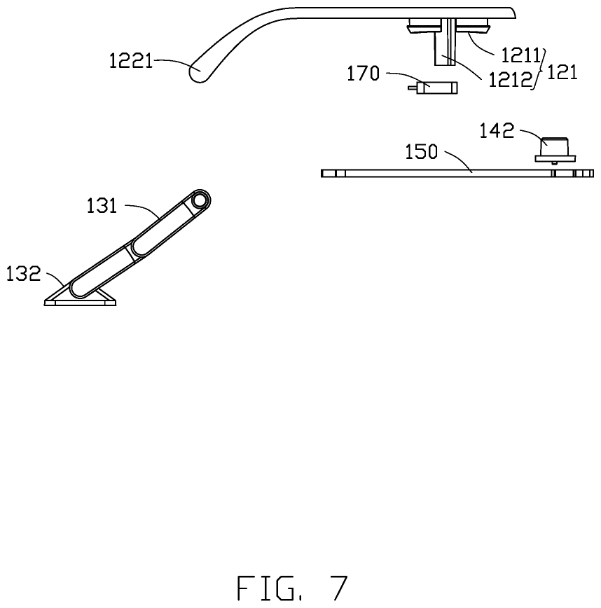

Referring to FIG. 7, a side view of the elements in FIG. 6 is provided, in accordance with an embodiment of the present disclosure. The distal part 122 has a bending shape, with the fork end 1221 bending towards the belt module 130. The central part 121 of the shaft 120 further comprises an axis 1212 extending downwardly from the coupling structure 1211 for coupling with the control module 150. The axis 1212 may be coupled to the opening 151 of the control module 150. Also, the axis 1212 may be configured to pass through the opening 151. The first sensor 170 is coupled to the axis 1213 of the shaft 130 and can be communicatively coupled to the control module 150. Specifically, the first sensor 170 can be installed on an upper side or a lower side of the control module 150. The axis 1212 is a center of a rotation of the shaft 120. When the shaft 120 is rotated, the first sensor 170 coupled to the axis 1212 will monitor a rotation angle of the shaft 120, then the rotation angle information will be transmitted to a controller (not shown) on the control module 150, The controller may process the rotation angle information and transmit the information to a computing apparatus. When the first joint function monitoring system 1 is worn by the subject and the shaft 120 is rotated along with the joint movement of the subject, the rotation angle represents a flexion or extension movement of the joint, therefore the physician or rehabilitation specialist may evaluate recovering progress or pathological conditions of the joint. The user may also evaluate a motor ability of the subject or conduct a functional diagnosis for the subject, when the first joint function monitoring system 1 is worn by the subject and the shaft 120 is rotated along with the joint movement. Thus, the joint function monitoring device 100 is able to measure and analyze joint movements in real-time by the first sensor 170 and the control module 150.

The computing apparatus may be a part of the control module 150, or another device that is not in contact with the assistive device 200 or the joint function monitoring device 100. The computing apparatus can he a central processing unit (CPU) or a microprocessing unit (MPU), or a device comprising the CPU or the MPU, Preferably, the computing apparatus is a mobile device.

The control module 150 comprises the controller, a switch (not shown), and a second sensor (not shown). The switch is connected to the button 142, and the user may press the button 142 to turn the joint function monitoring device 100 on or off. The button 142 may also connect to other functions embedded in the control module 150, such as mode-switching or recording. The second sensor on the control module 150 measures a tilting angle of the joint function monitoring device 100 when it is attached on the assistive device 200. The tilting angle is an angle between the assistive device 200 and the gravity on the coronal plane, whereas the extension and flexion are movements of the assistive device 200 on the sagittal plane.

The joint function monitoring device 100 comprises two links 131 in FIGS. 6 and 7, but the joint function monitoring device 100 may also comprise only one link 131. The amount of the links 131 can be from one to six. The more links 131 represents longer belt module 130. This configuration enables the belt module 130 to have an adjustable length. Because assistive devices may differ in shape and size, therefore the joint function monitoring device 100 having adjustable length is able to be attached on assistive devices of different shape and size. The adjustability of the device 100 enables the physician or rehabilitation specialist to devise a flexible rehabilitation plan for the subject. If the user wishes to retrieve information of the rotation angle of the joint from the device 100 of the present disclosure, the subject may remain wearing their original assistive devices, such as knee brace or hip brace, and attach the device 100 onto the original assistive devices. In this way, the equipment cost for the subject would be much lower than purchasing a brand new assistive device, and the subject will have a higher compliance when wearing the original device than switching to the brand new assistive device.

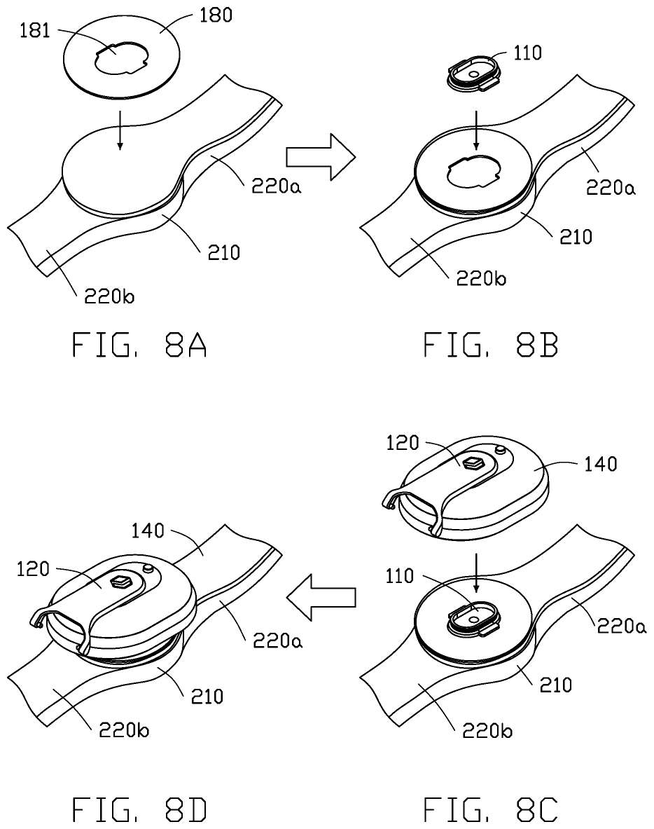

Referring to FIGS. 8A-8D, a first part of an installation process of the joint function monitoring device 100 is provided in accordance with an embodiment of the present disclosure. In FIG. 8A, a locator 180 is provided to be placed onto the hinge 210. The locator 180 can be a piece of cardboard or plastic board having an edge corresponding to the shape of the hinge 210. The locator 180 comprises an opening 181, and the opening 181 has a compound shape of 2 semi-circle combined with a rectangle, therefore the opening 181 has a longitudinal axis. When placing the locator 180 onto the hinge 210, two ends of the longitudinal axis of the opening 181 must be aligned with directions of the support assemblies 220a and 220b of the assistive device 288.

In FIG. 8B, the bottom base 110 has a shape that is identical to the opening 181. The user has to press the bottom base 110 when it is placed onto the opening 181, therefore a bottom side of the bottom base having adhesives is attached onto the hinge 210. In FIG. 8C, the modular piece (as illustrated in FIG. 5) is combined with the bottom base 110, Specifically, the bottom 144 of the modular piece is coupled to the bottom base 110. In FIG. 8D, when the modular piece is installed on the hinge 210, the locator 180 can be beneath the bottom 144. In FIG. 8D, the locator 180 can be removed before the bottom 144 of the modular piece is combined with the bottom base 110.

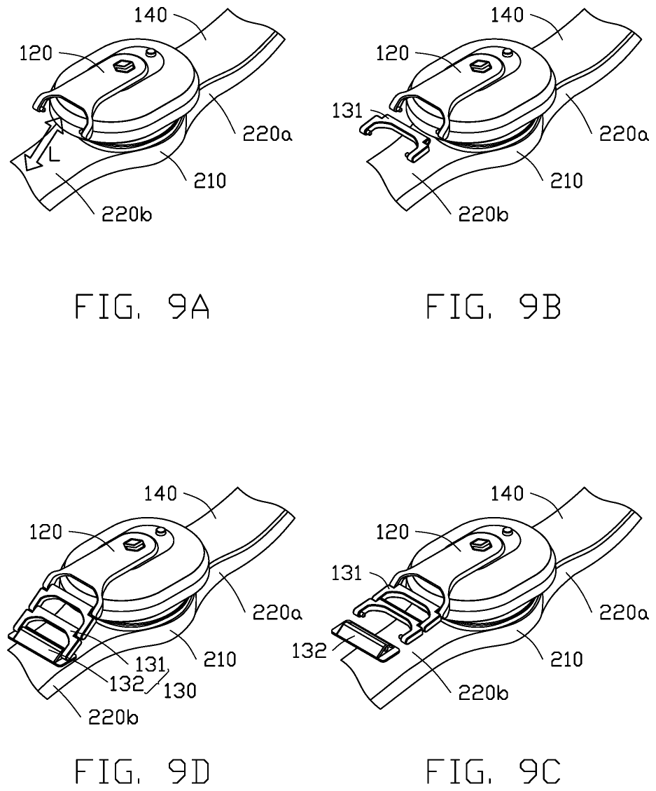

Referring to FIGS. 9A-9D, a second part of the installation process of the joint function monitoring device 100 is provided in accordance with an embodiment of the present disclosure. In FIG. 9A, a distance L between the shaft 120 and the supply assembly 220b is measured by the user to determine an amount of the links 131 to be coupled to the shaft 120. In FIGS. 9B and 9C, the links 131 and the link base 132 are provided. More than one link 131 may be needed to lengthen the belt module 130. The links 131 are first coupled to the distal part 122 of the shaft 120, the link base 132 are then coupled to one of the links 131 in FIG. 9D. The link base 132 comprises a bottom side with adhesive. In FIG. 9D, the link base 132 is pressed by the user, the adhesive on the bottom side enables the attachment of the link base 132 to the support assembly 220b. The installation of the joint function monitoring device 100 onto the assistive device 200 is thus, completed.

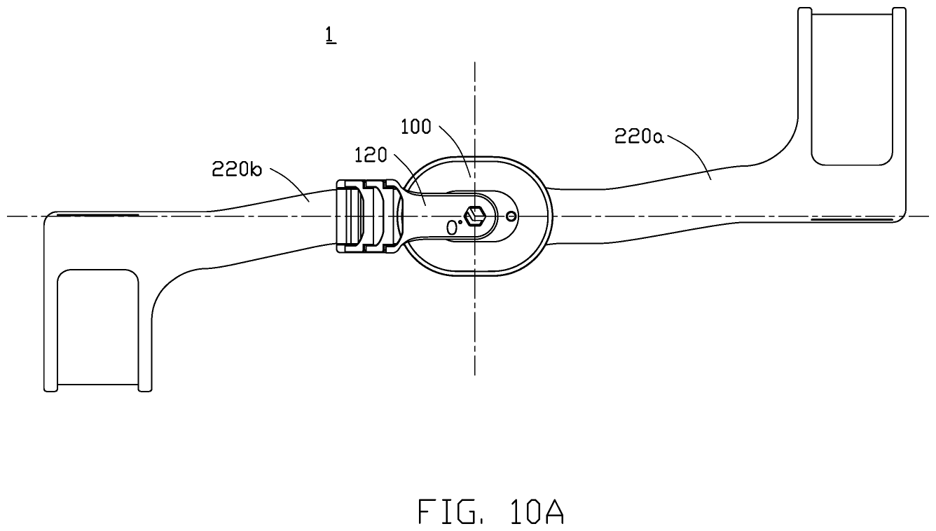

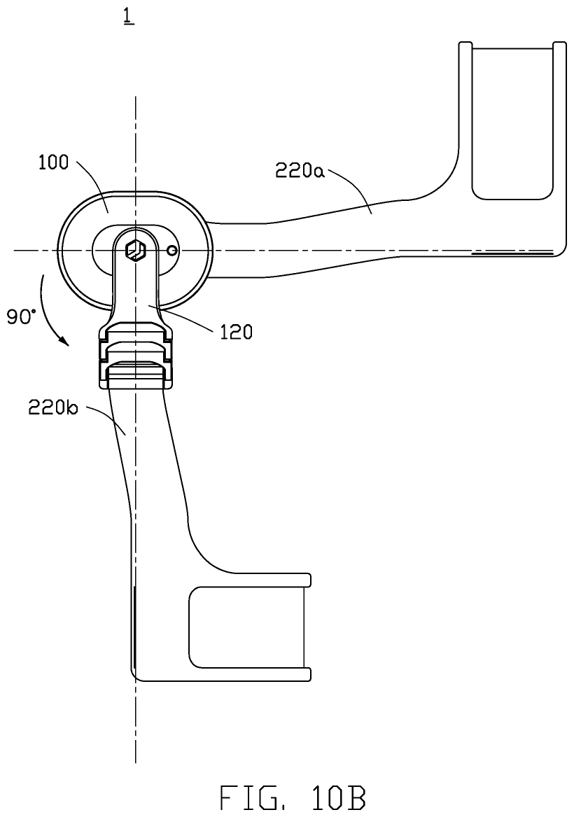

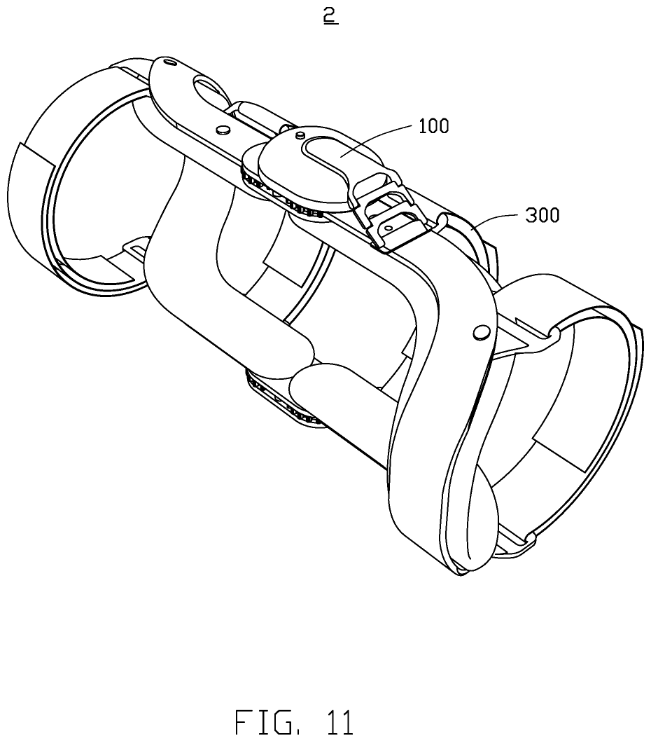

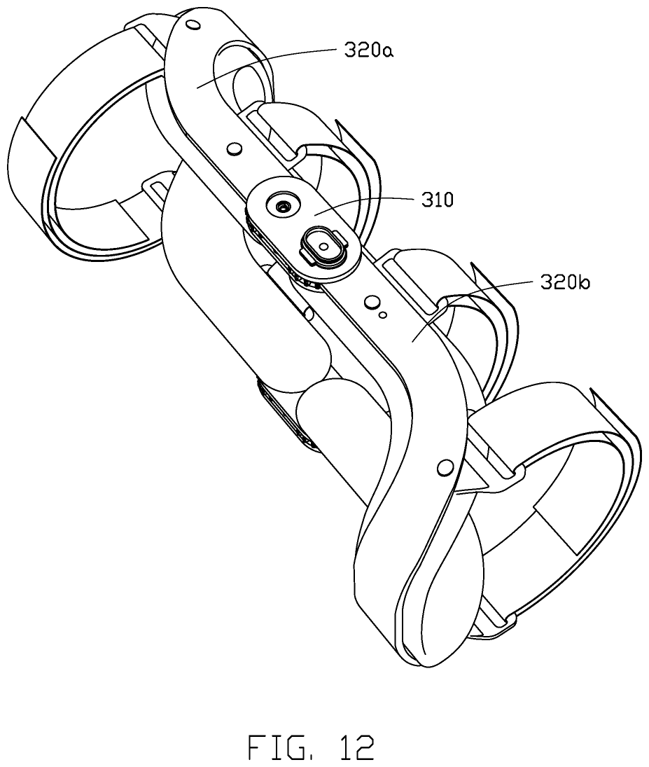

Referring to FIG. 10A, an extension of the first joint function monitoring system 1 is provided in accordance with an embodiment of the present disclosure. In FIG. 10A, an angle between the support assembly 220a and 220b is 0°. An extension angle of 0° can be monitored by the first sensor 170 in the joint function monitoring device 100.

Referring to FIG. 10B, a flexion of the first joint function monitoring system 1 is provided in accordance with an embodiment of the present disclosure. In FIG. 10B, the support assembly 220a is bended for 90°. When the subject is wearing the first joint function monitoring system 1, the bending of the support assembly 220a is most likely achieved by a flexion of the leg of the subject. The shaft 120 is rotated but the housing 140 is remained on the same direction and stationary relative to the hinge 210. A flexion angle of 90° is monitored by the first sensor 170 of the joint function monitoring device 100 and transmitted to the computing apparatus. The computing apparatus or the user is then able to compare the flexion angle and the extension angle and evaluate the function of the joint.

Referring to FIG. 11, a second joint function monitoring system 2 is provided in accordance with an embodiment of the present disclosure. The second joint function monitoring system 2 comprises an assistive device 300 and the joint function monitoring device 100 removably attached to the assistive device 300. The assistive device 300 and the assistive device 200 can be of different size and shape, however both of them could be removably attached by the joint function monitoring device 100. The joint function monitoring device 100 is able to be installed onto different assistive devices, this reduces the cost of joint rehabilitation for the subject wherein the subject would not need to switch to a different assistive device when using the joint function monitoring device 100 of the present disclosure. The modular design of the joint function monitoring device 100 also provides greater flexibility for the subject for it could be installed on assistive devices of various shapes and sizes.

Referring to FIG 12, a perspective view of the assistive device 300 is provided in accordance with an embodiment of the present disclosure. The assistive device 300 can be worn by the mammalian subject, and corresponds to shapes of the joint and body parts adjacent to the joint of the mammalian subject. The joint can be an ankle joint, a knee joint, a hip joint, a shoulder joint, an elbow joint, or a wrist joint. The body parts adjacent to the joint can be a foot, a leg, a hip, a shoulder, an arm, or a hand. The assistive device 300 is able to assist movements of the joint and the adjacent body parts, such as extension, flexion, or rotation. The assistive device 300 can be an ankle orthosis when the joint is the ankle joint, a knee orthosis when the joint is the knee joint, a hop orthosis when the joint is the hip joint, a shoulder orthosis when the joint is the shoulder joint, or a wrist orthosis when the joint is the wrist joint. Preferably, the assistive device 300 is a knee brace.

The assistive device 300 comprises a hinge 310 capable of being removably attached by the joint function monitoring device 100, and support assemblies 320a and 320b coupled to an upper part and a lower part of the hinge 310. The support assemblies 320a and 320b are fixation elements of the assistive device 300, and may comprise bars or bands. The support assemblies 320a and 320b may be attached to the body part adjacent to the joint by Velcro®, buttons, or straps, therefore the assistive device 300 can be firmly worn by the mammalian subject. The support assemblies 320a and 320b can be made of soft, semi-rigid, or rigid materials that is durable but also able to sustain persistent contact with the subject's skin. The hinge 310 provides extension or flexion movements within the assistive device 300. When the assistive device 300 is worn by the mammalian subject, the hinge 310 is aligned with the joint. Thus, when the mammalian subject bends the joint, the assistive device 300 is bent, and the support assemblies 320a and 320b will not form a straight line as illustrated in FIG 12.

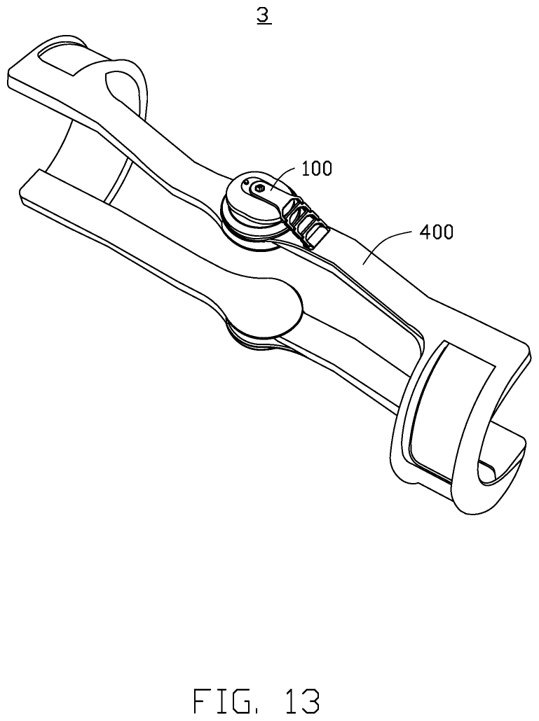

Referring to FIG 13, a third joint function monitoring system 3 is provided in accordance with an embodiment of the present disclosure. The third joint function monitoring system 3 comprises an assistive device 400, and the joint function monitoring device 100 removably attached to the assistive device 400. The assistive device 400, the assistive device 300, and the assistive device 200 can be of different size and shape, however they amid be removably attached by the joint function monitoring device 100. Specifically, the joint function monitoring device 100 is attached to the assistive device 400 by 3 of the links 131 and the link base 132 in the third joint function monitoring system 3, whereas the joint function monitoring device 100 is attached to the assistive device 300 by 2 of the links 131 and the link base 132 in the joint function monitoring system 2. This variable-link configuration enables the joint function monitoring device 100 to have different lengths, and can be flexibly attached to assistive devices of different shapes or sizes.

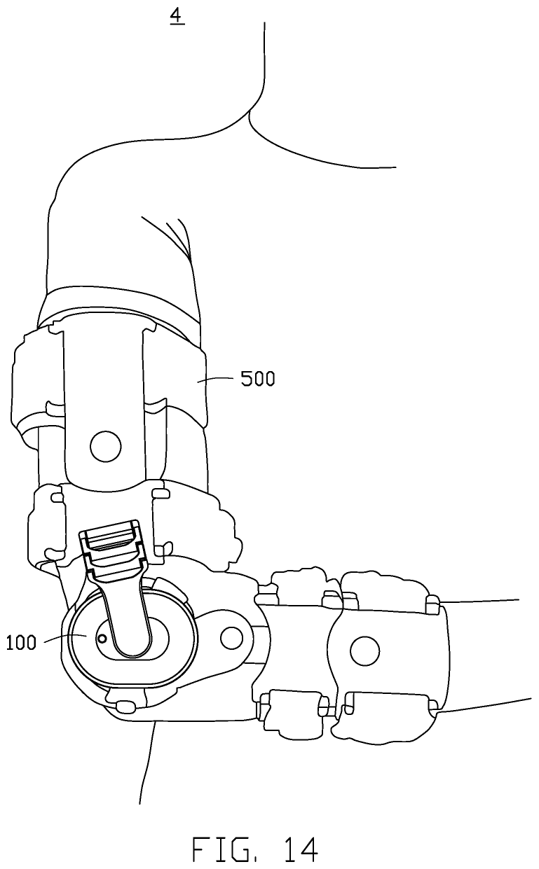

Referring to FIG. 14, a fourth joint function monitoring system 4 worn by the subject is provided in accordance with an embodiment of the present disclosure. The fourth joint function monitoring system 4 comprises an assistive device 500 and the joint function monitoring device 100 removably attached to the assistive device 500. The assistive device 500 can be worn on a different body part than the body parts being worn on the assistive device 400, 300, and 200. The assistive device 500 can be an orthosis for the elbow joint. The assistive device 500 is worn by the subject and corresponds to the shape of the elbow joint and an arm of the subject.

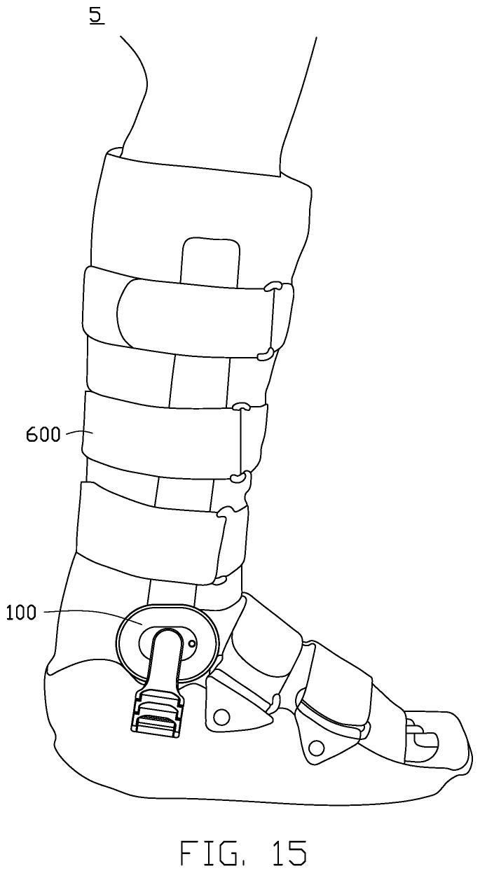

Referring to FIG 15, a fifth joint function monitoring system 5 worn by the subject is provided in accordance with an embodiment of the present disclosure. The fifth joint function monitoring system 5 comprises an assistive device 600 and the joint monitoring device 100 removably attached to the assistive device 600. The assistive device 600 can be worn on a different body part than the body parts being worn on the assistive device 500, 400, 300, and 200. The assistive device 600 can be an orthosis for the ankle joint. The assistive device 600 is worn by the subject and corresponds to the shape of the ankle joint, a leg and a foot of the subject. FIG 14 and 15 has demonstrated the flexibility, of the joint function monitoring device 100: it can be installed onto assistive devices for the elbow joint or the ankle joint.

The rotation angle of the shaft 120 can be from 0° to 180° when the first joint function monitoring system 1, the second joint function monitoring system 2, or the third joint function monitoring system 3 is worn on the knee joint of a bipedal mammalian subject. The rotation angle of the shaft 120 can also be from 0° to 180° when the fourth joint function monitoring system 4 is worn on the shoulder joint, or when the fifth joint function monitoring system 5 is worn on the ankle joint. However, the rotation angle of the shaft 120 can be from 0° to 360′ when the joint function monitoring device 100 is attached on another rehabilitation device that can be worn on other joints of the bipedal mammalian subject, or another rehabilitation device worn by other subjects having a joint that is capable of rotating for 360°. The shoulder joint of human is capable to rotate 360°, therefore the rotation angle of the shaft can be from 0 to 360° when the joint function monitoring device 100 is attached on a shoulder orthosis and the above combination is worn on the shoulder joint of a human subject.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is

1. A joint function monitoring device, comprising:
a bottom base capable of being removably attached to a hinge of an assistive device;
a shaft comprising a central part coupled to the bottom base, and a distal part;
a first sensor coupled to the central pan of the shaft for monitoring a rotation angle of the shaft; and
a belt module coupled to the distal part of the shaft and capable of being removably attached to a support assembly of the assistive device,
wherein a length of the belt module is adjustable.

2. The joint function monitoring device of claim 1, wherein the belt module comprises one or more links and a link base on a distal end of the belt module, the link base is coupled to at least one of the links and the support assembly of the assistive device, and at, least one of the links is removably coupled to the distal part of the shaft.

3. The joint function monitoring device of claim 2, wherein an amount of the links is in a range of one to six.

4. The joint function monitoring device of claim 1, further comprising a housing coupled to the bottom base, and a control module accommodated by the housing.

5. The joint function monitoring device of claim 4, wherein the control module further comprises a controller communicatively coupled to the first sensor, and a second sensor communicatively coupled to the controller.

6. The joint function monitoring device of claim 4, further comprising a locator, wherein when the bottom base is attached to the hinge of the assistive device, the locator is beneath the housing and aligned with the hinge.

7. The joint function monitoring device of claim 1, wherein the shaft further comprises an axis on the central part, and a center of the rotation of the shaft is the axis, and the rotation angle is about 0°-360°.

8. The joint function monitoring device of claim 7, wherein the axis is coupled to the first sensor.

9. A joint function monitoring system, comprising:
an assistive device comprising:
a hinge capable of being aligned with a joint of a mammalian body; and
a support assembly capable of being attached to a part of the mammalian body adjacent to the joint; and
a joint function monitoring device comprising:
a bottom base removably attached to the hinge of the assistive device;
a shaft comprising a central part coupled to the bottom base, and a distal part;
a first sensor coupled to the central part of the shaft for monitoring a rotation angle of the shaft; and
a belt module coupled to the distal part of the shaft and capable of being removably attached to the support assembly of the assistive device,
wherein a length of the belt module is adjustable.

10. The joint function monitoring system of claim 9, wherein the belt module comprises one or more links and a link base on a distal end of the belt module, the link base is coupled to at least one of the links and the support assembly of the assistive device, and at least one of the links is removably coupled to the distal part of the shaft

11. The joint function monitoring system of claim 10, wherein an amount of the links is in a range of one to six.

12. The joint function monitoring system of claim 9, wherein the joint function monitoring device further comprises a housing coupled to the bottom base, and a control module accommodated by the housing.

12. joint function monitoring system of claim 12, wherein the control module further comprises a controller communicatively coupled to the first sensor, and a second sensor communicatively coupled to the controller.

14. The joint function monitoring system of claim 12, wherein the joint function monitoring device further comprises a locator, wherein when the bottom base is attached to the hinge of the assistive device, the locator is beneath the housing and aligned with the hinge.

15. The joint function monitoring system of claim 9, wherein the shaft further comprises an axis on the central part, and a center of the rotation of the, shaft is the axis, and the rotation angle of the shaft is about 0°-360°.

16. The joint fiction monitoring system of claim 15, wherein the axis is coupled to the first sensor.

17. The joint function monitoring system of claim 9, wherein the joint is an ankle joint, a knee joint, a hip joint, a shoulder joint, an elbow joint, or a wrist joint.

18. The joint function monitoring system of claim 17, wherein the assistive device is an ankle orthosis when the joint is the ankle joint, a knee orthosis when the joint is the knee joint, a hip orthosis when the joint is the hip joint, a shoulder orthosis when the joint is the shoulder joint, or a wrist orthosis when the joint is the wrist joint.

19. The joint function monitoring system of claim 9, wherein the part of the mammalian body adjacent to the joint can be a foot, a leg a hip, a shoulder, an arm, or a hand.
